(19) United States
(12) Patent Application Publication
(10) Pub. No.: US 2025/0242048 A1
(43) Pub. Date: Jul. 31, 2025

(54) PARTICLE-BOUND LIPID NANOPARTICLE, METHOD OF PREPARATION, KIT, AND COMBINATION COMPOSITION

(71) Applicant: KABUSHIKI KAISHA TOSHIBA, Tokyo (JP)

(72) Inventors: Shu OKUMURA, Oyama (JP); Mitsuko ISHIHARA, Setagaya (JP)

(73) Assignee: KABUSHIKI KAISHA TOSHIBA, Tokyo (JP)

(21) Appl. No.: 19/033,705

(22) Filed: Jan. 22, 2025

(30) Foreign Application Priority Data
Jan. 24, 2024 (JP) 2024-008838
Nov. 8, 2024 (JP) 2024-195648

(51) Int. Cl. A61K 47/69; A61K 47/66; B82Y 5/00

(52) U.S. Cl. CPC A61K 47/6911; A61K 47/665; B82Y 5/00

(57) ABSTRACT

According to one embodiment, a particle-bound lipid nanoparticles includes a biodegradable lipid nanoparticle, a particle bound to an outer surface of the lipid nanoparticle, and an active substance bound to a surface of the particle.

### CROSS-REFERENCE TO RELATED APPLICATIONS

This application is based upon and claims the benefit of priority from Japanese Patent Application No. 2024-008838, filed Jan. 24, 2024; and No. 2024-195648, filed Nov. 8, 2024, the entire contents all of which are incorporated herein by reference.

### FIELD

Embodiments described herein relate generally to particle-bound lipid nanoparticles, method of preparation, kit and combination composition.

### BACKGROUND

Lipid nanoparticles are known as a means of delivering desired active substances, such as therapeutic genes or drugs, into the target cells. Such lipid nanoparticles encapsulate the active substance to be delivered into a capsule composed of a lipid membrane. The lipid nanoparticles typically release the active substance into the target cell through pathways such as membrane fusion or endocytosis.

On the other hand, TRAIL receptors have attracted attention as a molecular target for cancer therapy. TRAIL (TNF-related apotosis-inducing ligand) is a cytokine involved in tumor immune surveillance mechanisms. Recently, it has been shown that the expression level of Lewis glycans, which are present on cancer cells and attached to cell lipids, is the key to promoting TRAIL receptor-mediated cancer cell death. Therapy using such a pathway to deliver active substances to the surface of the cell membrane requires that the active substances remain on the surface of the cell membrane, which is difficult to achieve in the conventional form of lipid nanoparticles which deliver active substances inside the cell.

### BRIEF DESCRIPTION OF THE DRAWINGS

FIG. 1 is a schematic view showing one example of a particle-bound lipid nanoparticle of a first embodiment.

FIG. 2 is a schematic view showing one example of the particle-bound lipid nanoparticle of the first embodiment.

FIG. 3 is a schematic view of a concept of an example of a manufacturing method of a second embodiment.

FIG. 4 is a schematic view of an example of the manufacturing method of the second embodiment.

FIG. 5 is a schematic view of an example of the embodiment.

FIG. 6 is a schematic view showing comparative example (a) and example (b) used in an experiment of example 1.

FIG. 7 is an image showing a result of experiment.

FIG. 8 is an image showing a result of experiment.

FIG. 9 is an image showing a result of experiment.

FIG. 10 is a schematic view showing comparative example (a) and example (b) used in an experiment of example 2.

FIG. 11 is an image showing a result of experiment.

FIG. 12 is an image showing a result of experiment.

### DETAILED DESCRIPTION

In general, according to one embodiment, particle-bound lipid nanoparticles include a biodegradable lipid nanoparticle, a particle, and an active substance. The particle is bound to the outer surface of the biodegradable lipid nanoparticle. The active substance is bound to the surface of the particle.

Hereinafter, embodiments will be explained with reference to the accompanying drawings. Note that, in each embodiment, same structural components will be referred to by the same reference numbers, and explanation thereof will be partially omitted. The figure is schematic, and the relationship between the thickness of each part and the plane dimensions, the ratio of the thickness of each part, etc., may differ from those in reality.

In the present application, “lipid nanoparticle” refers to a particle which are mainly constituted by lipids. For example, forms commonly referred to as lipid nanoparticles (LNPs), micelles, liposomes, and micro-emulsions are also included in the term “lipid nanoparticle” herein.

### First Embodiment

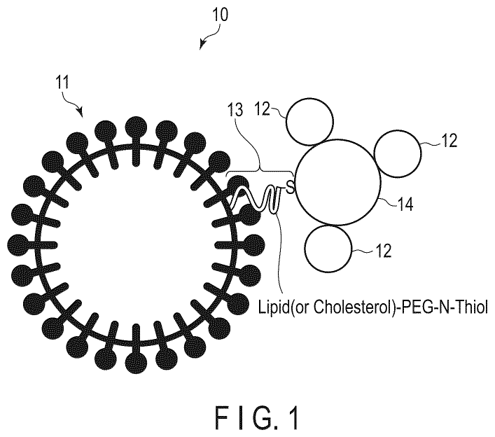

Particle-bound lipid nanoparticles according to an embodiment will be described referring to FIG. 1. A particle-bound lipid nanoparticle 10 includes a biodegradable lipid nanoparticle 11, active substance 12, and particle 14. The particle 14 is bound to the outer surface of lipid nanoparticle 11. The active substance 12 is bound to the surface of the particle 14. As to the binding of the lipid nanoparticle 11 to the particle 14, for example, the particle 14 may be directly bound to a portion of the lipids comprising the lipid nanoparticles 11, or may be bound to the lipid nanoparticles 11 via a linker 13 of the lipid nanoparticle 11. For example, the linker 13 can be a linker structure known per se which is attached to and/or extends from a functional group of any of the lipids of the lipid nanoparticle 11. For example, the linker 13 can be a portion of any lipid of the biodegradable lipid nanoparticle 11, such as a portion of a PEG-modified lipid, a portion of a lipid provided with a functional group capable of binding a ligand, etc. For example, the linker 13 can be a PEG-modified group with a thiol group at the end. FIG. 1 shows an example of binding to cholesterol as an example of the lipid bound with a modified group. However, the linker 13 is not limited to such a case. As will be described in detail below, any one or more PEG-modified lipids, lipids with functional groups capable of binding ligands, and the like may be included as lipids constituting the lipid nanoparticle 11. When the lipid nanoparticles 11 are formed using these lipids, a portion of those lipids or modified groups may be utilized as the linker 13. Examples of functional groups to which the ligand can be bound include, but are not limited to, active ester groups, thiol groups, amino groups, maleimide groups, and carboxy groups. Also, for example, the linker 13 can be included in the lipid material or formed lipid nanoparticles 11 at a mole fraction of 0.01 to 1.0%. The linker 13 and particles 14 can be used to stably bind the particles 14 to the lipid nanoparticles 11 and position them outside of the lipid nanoparticles 11.

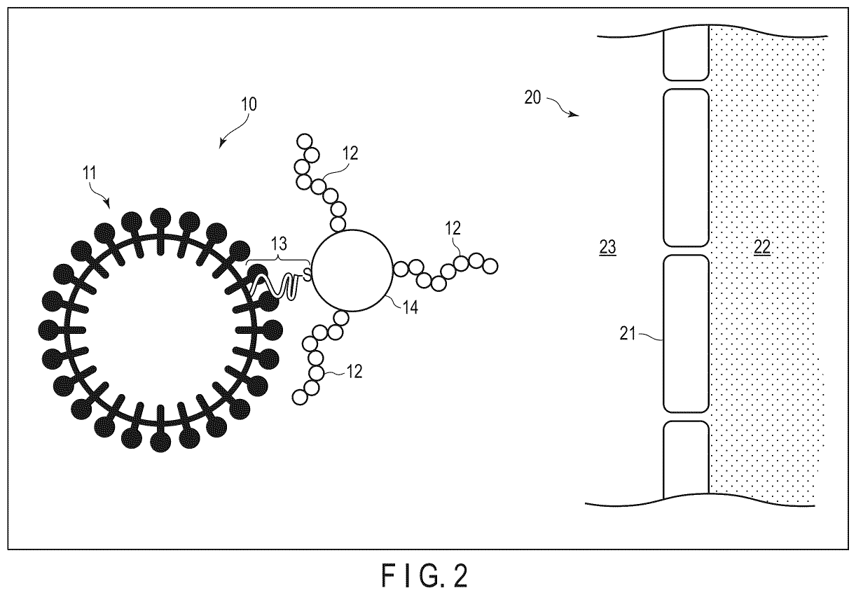

For example, the mode of binding of the linker 13 to the particle 14 should be determined according to the material of the particle and/or the configuration of the linker 13. As mentioned above, FIG. 1 shows one example in which a thiol group is present at the outer terminus of the linker 13, but is not limited thereto, and functional groups such as amino groups, active ester groups, maleimide groups, carboxy groups, etc., may be utilized. On the other hand, the particles 14 are each composed of materials which can bind or have affinity to the linker 13. For example, the particles 14 may be composed of a material which is capable of or has affinity for the thiol group and capable of or has affinity for the desired active substance 12. Such particles 14 may, for example, be selected from the group consisting of gold, silver, iron oxides, titanium oxides, zinc oxides, silica, lipid nanoparticles, polymer particles, and combinations of any two or more thereof, and may be mainly composed thereof. The surface of the particles may be physically treated to facilitate binding to the linker and/or immobilization or binding of the radioactive material. Such surface treatment is, for example, hydrophilic. The particles are cell-surface-entangled microparticles, in other words, they have a diameter that does not, for example, pass through cell membranes. An image of a cell-surface-residing microparticle-bound lipid nanoparticle applied to a cell is shown in FIG. 2. The particles 14 do not pass through the cell membrane 21 of the cell 20 and do not migrate into the cell 22, but remain on the outside 23 of the cell membrane. In other words, the particles 14 have the property of remaining on the cell membrane for a certain amount of time. This property is related to the size of the particle, and thus, for example, the particle should be of a size that does not permeate the cell membrane or is less permeable to the cell membrane. The fixed time may be the time required for the active substance on the particles to act, e.g., 10 minutes, 1 hour, 1 day, or 1 week.

For example, the diameter of the particles should be between 1 nm and 10 μm. Even more desirable is 10 nm to 5 μm, and even more desirable is 100 nm to 5 μm. For some cells, if the particle size is too small, uptake by endocytosis and the like may be enhanced, making it difficult for the particles to stay on the cell membrane, so it is preferable to select an appropriate particle size in each case. For example, one or more active substances 12 are bound, attached or immobilized on the surface of such particles 14; in the example of FIG. 1, three active substances 12 are immobilized on the surface of particles 14, but this is not limited thereto. The active substances 12 bound to one lipid nanoparticle 11 may be one type or a combination of two or more types. In other words, the active substance 12 bound to one lipid nanoparticle 11 may comprise one type of component or a plurality of components of different types from each other. Or, if multiple cell surface-staying particle-bound lipid nanoparticles 11 are used for one system in which target cells to be targeted are present, different types of active substances 12 may be selected and combined among the lipid nanoparticles 11. For example, the form of the active substance 12 is described schematically as a circle or sphere in FIG. 1, but is not limited thereto. For example, the molecules may be represented as chains or strings as shown in FIG. 2, and may be in any form depending on the substance.

The active substance 12 may be a synthetic or natural substance. Examples of active substances 12 can be active substances which target, for example, cell surfaces and/or molecules present on the cell surface, structures on the cell surface, etc. Examples of such substances are, for example, active substances which have effects on specific receptors, channels, surface structures, etc., or through which they have the effects on the target cell, including, for example, substances having any pharmacological effect, receptor antagonists, receptor inhibitors, receptor agonists, anticancer agents, etc.

A “target cell” may be a cell on which the active substance is to act. For example, the target cell may be a cell with a phospholipid bilayer or a cell-like structure, e.g., a cell with a cell membrane. For example, it can be a cell of animal or bacterial origin. It can also be, for example, a cancer cell, a proliferating cell, a cell affected by any other disease or a damaged cell. Examples of cancer cells are metastatic cancer, blood cancers such as leukemia, ovarian cancer, thyroid cancer, pheochromocytoma, multiple myeloma, melanoma, glioma, leukemia, prostate cancer, breast cancer, etc. For example, at the laboratory level, normal cells can be target cells. For example, the target cells may be selected according to the type of active substance used, the type or characteristics of the biodegradable lipid nanoparticles, the therapeutic target and/or the wishes of the practitioner. Application of particle-bound lipid nanoparticles to target cells may be, for example, clinical, laboratory, in vivo, in vitro, systemic, local, or any suitable route, such as intravascular, intraperitoneal, or organ administration.

The biodegradable lipid nanoparticles 11 should have target cell tropism. Here, “target cell tropism” means, for example, having an appropriate affinity for the target cell. Herein, “appropriate affinity” means having a higher affinity compared to similar transfection carriers of general design under normal and/or general contact conditions with the target cell and/or having a higher affinity for the target cell compared to affinity for cells other than the target cell. Target cell tropism, i.e., appropriate affinity, is achieved by adjusting the lipid composition of the biodegradable lipid nanoparticles.

The biodegradable lipid nanoparticles 11 include a lipid composition which exhibits the desired target cell tropism. The biodegradable lipid nanoparticle 11 is a sphere or abbreviated sphere formed of a lipid membrane, in other words, a hollow lipid nanoparticle. It can be a lipid membrane particle which encapsulates a core of aqueous solution, e.g., a lipid bilayer membrane particle. The lipid nanoparticle may be any publically-known lipid nanoparticle. For example, the lipid composition of the lipid nanoparticles may include, as its components, a first lipid (FFT-10) of formula (I) and/or a second lipid (FFT-20) of formula (II). These lipids are biodegradable lipids. By adjusting the lipid composition of the biodegradable lipid nanoparticles with these lipids, an appropriate affinity may be achieved.

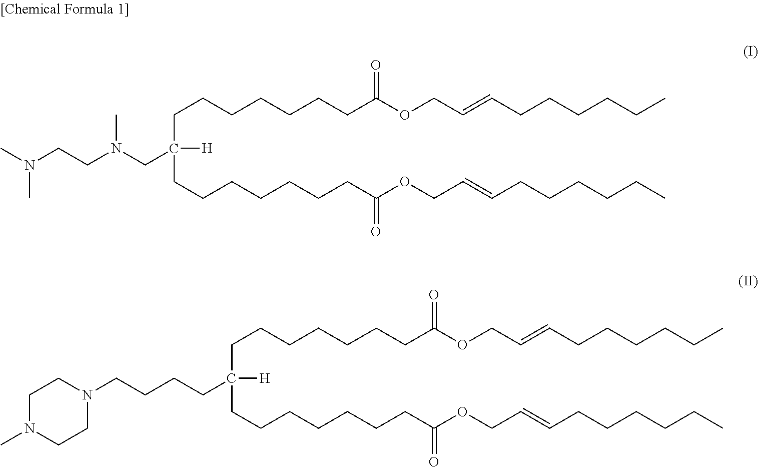

The biodegradable lipid nanoparticles, i.e., lipid nanoparticles, may contain further lipids in addition to the first and second lipids described above. Among the composition of the lipid molecular materials constituting the lipid nanoparticles, the fraction consisting of the first and second lipids is hereinafter referred to as “first fraction”. The fraction consisting of lipid molecular materials other than the first or second lipid is hereinafter referred to as “second fraction”. The lipids in the second fraction are hereinafter also referred to collectively as “third lipid”.

The terms first and second fractions refer to the composition of the constituents of the lipid nanoparticles, not to the physical location of the lipids contained therein. For example, the components of the first and second fractions need not each be in one cohesive mass in the lipid nanoparticle, and the lipids in the first fraction can exist intermixed with those in the second fraction. The ratio of the first fraction to the total lipid material of the lipid nanoparticles can be 10% or more, 15% or more, 20% or more, 30% or more, 40% or more, 50% or less, 40% or less, 30% or less, 20% or less, for example, 10 to 50%, or 15 to 45%.

In other words, the total content of FFT-10 and/or FFT-20 as a percentage of lipid nanoparticles may be, for example, 10 to 50%, 10 to 45%, 10 to 40%, 10 to 35%, 10 to 30%, 10 to 25%, 10 to 20%, 15 to 50%, 15 to 45%, 15 to 40%, 10 to 35%, 15 to 30%, 15 to 25%, 15 to 20%, 20 to 50%, 20 to 45%, 20 to 40%, 20 to 35%, 20 to 30%, or 20 to 25%. The maximum content of FFT-10 and FFT-20 in the lipid nanoparticles may be, for example, the amount by which the lipid nanoparticles can form lipid nanoparticles. The percentage of the second lipid in the first fraction may be from 0 to 100%, for example, 15 to 75%, 20 to 60%, or 24 to 50%. Similarly, the percentage of the first lipid in the first fraction may be from 0% to 100%, for example, 15 to 75%, 20 to 60%, or 24 to 50%. Herein, percentages are expressed in mol/mol % unless otherwise specified.

The particle size and cell penetration of the lipid nanoparticles may change depending on the ratio of the first lipid to the second lipid in the first fraction. For example, when the second lipid increases, the particle size of the lipid nanoparticles may become larger. The average particle size of the lipid nanoparticles can be changed depending on the application. For example, it may be adjusted from about 20 to 300 nm. For example, it may be from about 50 to 100 nm.

The type of third lipid in the second fraction of lipid nanoparticles is not limited, but for example, the second fraction contains base lipids. The base lipid can be, for example, a lipid which is a major component of biological membranes. The base lipid may be a phospholipid or sphingolipid, such as diacylphosphatidylcholine, diacylphosphatidylethanolamine, ceramide, sphingomyelin, dihydrosphingomyelin, kephalin or cerebroside, or a combination thereof.

For example, the base lipid is the following:
1,2-dioleoyl-sn-glycero-3-phosphoethanolamine (DOPE),
1,2-stearoyl-sn-glycero-3-phosphoethanolamine (DSPE),
1,2-dipalmitoyl-sn-glycero-3-phosphatidylcholine (DPPC),
1-palmitoyl-2-oleoyl-sn-glycero-3-phosphatidylcholine (POPC),
1,2-di-O-octadecyl-3-trimethylammonium propane (DOTMA),
1,2-dioleoyl-3-dimethylammonium propane (DODAP),
1,2-dimyristoyl-3-dimethylammonium propane (14:0 DAP),
1,2-dipalmitoyl-3-dimethylammonium propane (16:0 DAP),
1,2-distearoyl-3-dimethylammonium propane (18:0 DAP),

N-(4-carboxybenzyl)-N,N-dimethyl-2,3-bis(oleoyloxy)propane (DOBAQ),
1,2-dioleoyl-3-trimethylammonium propane (DOTAP),
1,2-dioleoyl-sn-glycero-3-phosphochlorin (DOPC),
1,2-dilinoleoyl-sn-glycero-3-phosphochlorin (DLPC),
1,2-dioleoyl-sn-glycero-3-phospho-L-serine (DOPS), or
Cholesterol, or any combination of the above is preferred. As the above base lipids, it is especially preferable to use cationic or neutral lipids, and the acid dissociation constant of the lipid nanoparticles can be adjusted by the content thereof. DOTAP is preferably used as the cationic lipid, and DOPE is preferably used as the neutral lipid.

The percentage of cationic lipids such as FFT10, FFT20, and DOTAP to the total lipid nanoparticles should be, for example, about 10 to 50% to adjust the appropriate affinity to the target cells. The percentage of cationic lipids to the total lipid nanoparticles is preferred to be, for example, 10 to 50%, 10 to 45%, 10 to 40%, 10 to 35%, 10 to 30%, 10 to 25%, 10 to 20%, 15 to 50%, 15 to 45%, 15 to 40%, 10 to 35%, 15 to 30%, 15 to 25%, 15 to 20%, 20 to 50%, 20 to 45%, 20 to 40%, 20 to 35%, 20 to 30%, and 20 to 25%. Adjustment of the lipid composition may be adjusted and designed to obtain an appropriate affinity for the target cell by, for example, changing the component ratio of the cationic lipid to be included in the lipid nanoparticles or by having a gradient, depending on the type and condition of the target cell. For example, the component ratio of the cationic lipid may be adjusted to have an appropriate affinity for the target cells in a particular state.

The second fraction also preferably contains lipids which prevent aggregation of lipid nanoparticles. For example, lipids which prevent aggregation include PEG-modified lipids, such as polyethylene glycol (PEG) dimyristoylglycerol (DMG-PEG), omega-amino (oligoethylene glycol) alkanoic acid monomer derived from polyamide oligomers (U.S. Pat. No. 6,320,017 B) or monosialogangliosides may be further included. The second fraction may further contain lipids such as relatively less toxic lipids to adjust for toxicity; lipids with functional groups which bind ligands to lipid nanoparticles; lipids to inhibit leakage of inclusions such as sterols, for example cholesterol. It is particularly desirable to include cholesterol.

The type and composition of the lipid used in the second fraction may be appropriately selected by considering the acid dissociation constant (pKa) of the target lipid nanoparticles, or the particle size of the lipid nanoparticles, or the type of active agent to be included, or stability in the cell.

One or more than one type of lipid from any of the above may be selected as desired for the linker 13. For example, the hydrophobic end of lipids such as DMG, DSPE, cholesterol, and DOPE may be modified by PEG or other modification to form the linker 13.

The linker 13 may be included in a mole fraction of 0.01 to 1.0% of the component molecules of the biodegradable lipid nanoparticles (e.g., the first, second, and third lipids described above, including the lipids of the first and second fractions). In other words, for example, if the amount of lipid used in a lipid nanoparticle is 1 mole, the amount of lipid of the linker 13 contained therein should be 0.0001 to 0.01 mole. Even more preferably, the linker should be included in a mole fraction of 0.3% of the component molecules of the biodegradable lipid nanoparticles.

For example, further components may be included within the biodegradable lipid nanoparticles. The further component may be, for example, a further active substance, a substance having other pharmacological activity, or a nucleic acid construct encoding a gene. Further components may also be encapsulated as needed. Such components may be, for example, pH adjusters, osmotic pressure regulators, and gene activators. The pH adjusters are, for example, organic acids such as citric acid and their salts. The osmotic pressure regulators are, for example, sugars or amino acids. The gene activators are, for example, any substance which promotes or supports the activity of the active agent if the further active agent is a gene. Alternatively, for example, the biodegradable lipid nanoparticles may encapsulate a labeling substance which enables the particle-bound lipid nanoparticles to be detected or visualized. For example, such a labeling substance may be a radioactive, fluorescent, dye, and chemiluminescent substance. For example, the above further substances and/or active agents may be one or a combination of two or more. Alternatively, such further substances and/or active agents may comprise a single component or a plurality of components of different types from each other.

As mentioned above, the particle-bound lipid nanoparticle according to the embodiment contains a biodegradable lipid nanoparticle, a particle, and an active substance. The particle is bound to the outer surface of the biodegradable lipid nanoparticle. The active substance is bound to the surface of the particle. The particle-bound lipid nanoparticle facilitates the active agent to act on the cell surface by staying on the cell surface. Conventionally, the lipid nanoparticles are used as a system to deliver the substance to the inside of a cell, for example, by encapsulating the desired substance therein. The present embodiment is based on the inventor's discovery that it is possible to deliver a target substance to the surface of a cell by purposely maintaining the target substance on the surface of lipid nanoparticles, which is a novel delivery system achieved by drastically unprecedented idea. By binding the active substance to the outer surface of the biodegradable lipid nanoparticles, such particle-bound lipid nanoparticles can be made to stay on the target cell surface more readily. The above mentioned embodiment can provide a novel technology for delivering the active substance as the active ingredient to target cells. It is also estimated to reduce side effects derived from the delivery system.

### Second Embodiment

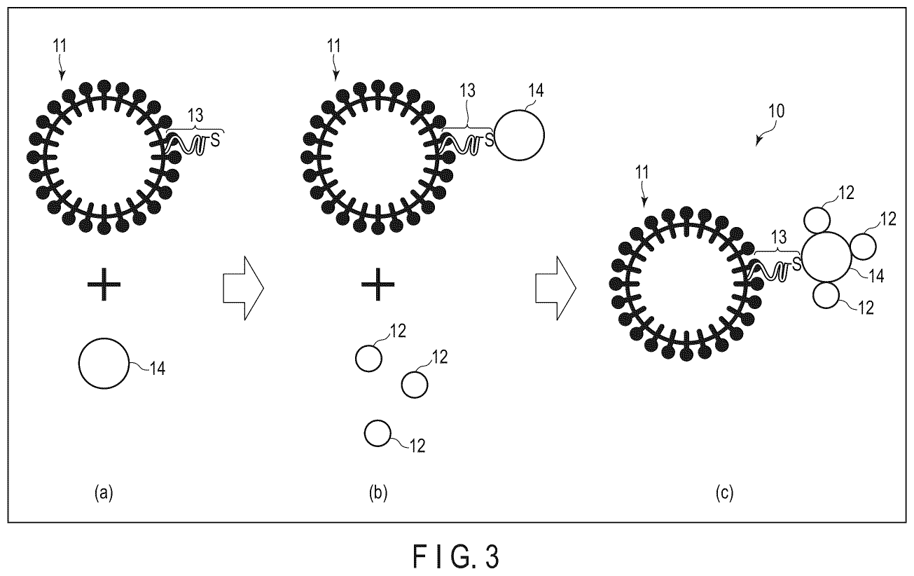

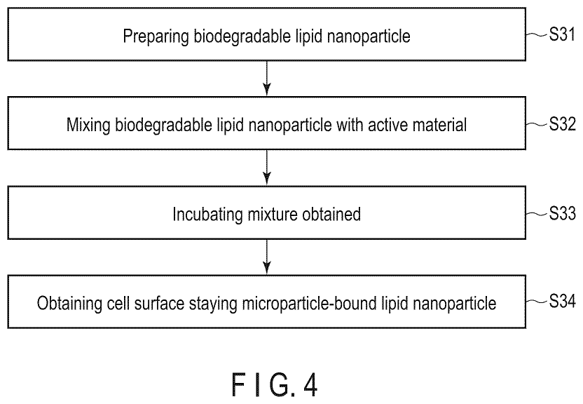

One example of a method of manufacturing the particle-bound lipid nanoparticles 10 with an active substance according to a second embodiment will be described referring to FIGS. 3 and 4. First, a lipid material having desired lipid composition is used to form lipid nanoparticles 11, for example, by the publically-known method as described above (part (a) of FIG. 3). At least a portion of the lipid material used herein should be modified such that the lipid nanoparticles 11 contain the desired linker 13, for example. The particles 14, which can be immobilized at the end of the linker 13, are then added and stirred for incubation (part (a) of FIG. 3). Furthermore, the active substance 12 is added, stirred and incubated (part (b) of FIG. 3). As a result, the particle-bound lipid nanoparticle 10 with active substance is obtained (part (c) of FIG. 3). The incubation may be performed, for example, by leaving them under a constant temperature. These processes can be rephrased as follows. That is, the manufacturing method of cell surface-staying microparticle-bound lipid nanoparticles 10 includes a preparing biodegradable lipid nanoparticle 11 (part (a) of FIG. 3, FIG. 4 (S31)), mixing the biodegradable lipid nanoparticle 11 and active substance 12 (part (b) of FIG. 3, FIG. 4 (S32)), and then mixing the resulting mixture incubating (part (a) of FIG. 3, FIG. 4 (S33)), and obtaining the particle-bound lipid nanoparticle 10 (part (c) of FIG. 3, FIG. 4 (S34)). Here, the microparticle 14 may be bound to the outer surface of the biodegradable lipid nanoparticles 11 at a time prior to the mixing of the biodegradable lipid nanoparticle 11 and the active substance 12 (part (b) of FIG. 3) (not shown), or they may be bound or immobilized with the active substance 12 prior to the above mixing (FIG. 3 (b)) (not shown).

The preparation of biodegradable lipid nanoparticle 11 may be performed, for example, by the formation of lipid nanoparticles using the desired materials by the Bangham method, organic solvent extraction, surfactant removal, or freeze-thaw method. For example, lipid nanoparticles may be formed by preparing a lipid mixture obtained by including the biodegradable lipid nanoparticle material in a desired ratio in an organic solvent such as alcohol and an aqueous buffer; adding the aqueous buffer to the lipid mixture; and stirring and suspending the resulting mixture. The lipid nanoparticles obtained as above are one example of biodegradable lipid nanoparticles 11. For example, encapsulation of further active agents or further components in the lipid nanoparticles 11 may be achieved by including the components to be encapsulated in the aqueous buffer solution described above.

For example, the conditions of incubation may be selected according to the nature of the active substance used and under pharmaceutically acceptable conditions. For example, incubation may be performed at temperature conditions of about 4 to 37° C. for about 10 minutes to about 1 hour. Alternatively, for example, incubation may be performed by leaving at room temperature.

Obtaining particle-bound lipid nanoparticles 10 containing the active substance will be satisfied when the desired particle-bound lipid nanoparticles 10 are formed. If desired, further steps may be included. For example, it may further include isolating the formed particle-bound lipid nanoparticles 10, and it may further include washing the resulting particle-bound lipid nanoparticles 10.

According to the manufacturing method of particle-bound lipid nanoparticle 10, it is possible to provide a new technology for delivering active substances as active components to the cell membrane surface of target cells. It is possible to more easily obtain target cell-directed lipid nanoparticles as a means of delivering the active substance to the target cell.

### Third Embodiment

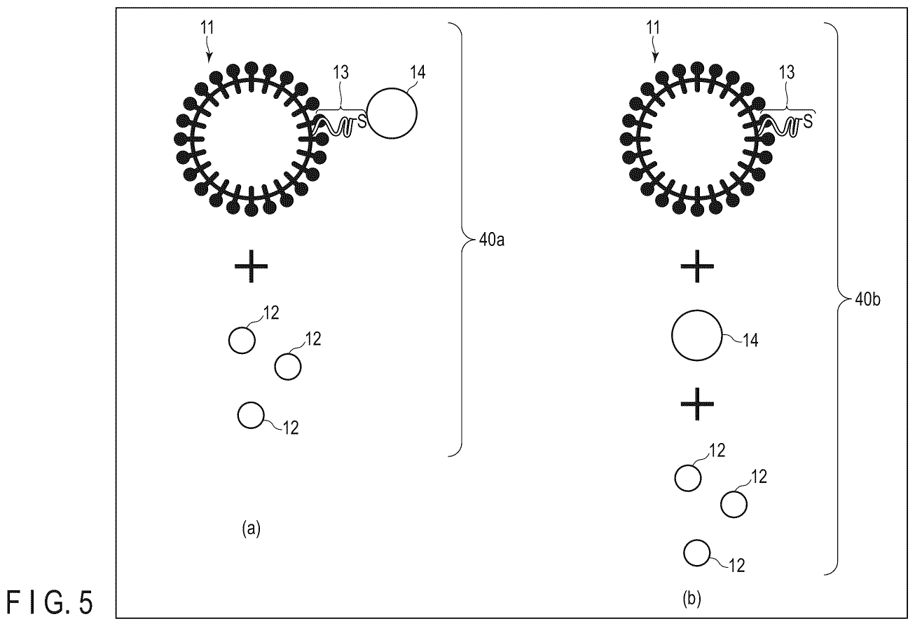

The particle-bound lipid nanoparticle according to the first embodiment described above may be provided ready for immediate use on the desired target cells, or may be provided as a particle-bound lipid nanoparticle manufacturing kit in the form of a material which can be adjusted at the time of use by the user of the particle-bound lipid nanoparticle described above. Specifically, the kit may be provided with biodegradable lipid nanoparticle 11 or biodegradable lipid nanoparticle material (not shown) configured to be directed to a desired target cell and an active substance 12 (part (a) of FIG. 5). Alternatively, a portion of the composition of the biodegradable lipid nanoparticle 11, e.g., particle 14, may be provided in a form independent of the lipid nanoparticle (part (b) of FIG. 5).

Providing the product as a kit for the preparation of particle-bound lipid nanoparticles facilitates handling in the appropriate environment for each configuration or component.

### Fourth Embodiment

The particle-bound lipid nanoparticles (e.g., cell surface-staying microparticle-bound lipid nanoparticles) according to the first embodiment described above and the particle-bound lipid nanoparticle manufacturing kit according to the second embodiment (e.g., cell surface-staying microparticle-bound lipid nanoparticle manufacturing kit) may be provided as a composition in a state ready for immediate use in a desired target cell (e.g., pharmaceutical composition) or as a combination composition which is adjusted immediately before use by a user of the particle-bound lipid nanoparticles (e.g., cell surface-staying microparticle-bound lipid nanoparticles) described above. For example, when provided as a combination composition, the combination composition may include, for example, a first composition with biodegradable lipid nanoparticles 11 configured to be directed to a desired target cell to which the micro particles 14 are externally bound and a second composition with an active substance 12 (part (a) of FIG. 5). Alternatively, it may include a first composition with the base lipid nanoparticles 11, a second composition with the active substance 12, and a third composition with the microparticles 14 (part (b) of FIG. 5). Furthermore, a lipid material (not shown) may also be provided as a composition to be combined, instead of the biodegradable lipid nanoparticles 11, to comprise the biodegradable lipid nanoparticles. The composition and combination composition may include particle-bound lipid nanoparticles (e.g., cell surface-staying microparticle-bound lipid nanoparticles) and may further include the desired components and/or composition (components and/or composition thereof may be publically-known). For example, the components and/or composition may be selected to provide physically and/or chemically stable lipid nanoparticles and/or particle preparation kits, pharmacologically and/or medically stable, or physically and/or chemically and/or pharmacologically and/or medically sufficient to meet the necessary and sufficient conditions.

The composition or combination composition may be a pharmaceutical composition. For example, the composition or combination composition includes a particle-bound lipid nanoparticle or particle-bound lipid nanoparticle material in a pharmaceutically acceptable state and/or as an ingredient for delivering an active substance as an active ingredient to the cell membrane surface of a target cell. Such compositions may be used in clinical or non-clinical fields. Such composition or combination composition may include appropriate additives, such as stabilizers, pH adjusters, buffers, viscosity adjusters, excipients, etc., depending on the desired conditions (method of use, route of administration, target cells to be used or subject to be administered), respectively. For example, when provided as a pharmaceutical composition to be administered to a subject, the ingredients included are selected and designed within a pharmaceutically acceptable range.

Such composition or combination composition could provide novel technologies for delivering active substances as active ingredients to the cell membrane surface of target cells. It is also predicted to reduce side effects derived from the delivery system.

### EXAMPLE

As an example of a particle-bound lipid nanoparticle according to the embodiment, an experimental example in which a drug delivery system which mimics a cell surface-staying microparticle-bound lipid nanoparticle was constructed, and target cell tropism thereof was examined will be explained.

### Experiment 1: Preparation of Target Cell-Directed Lipid Nanoparticles

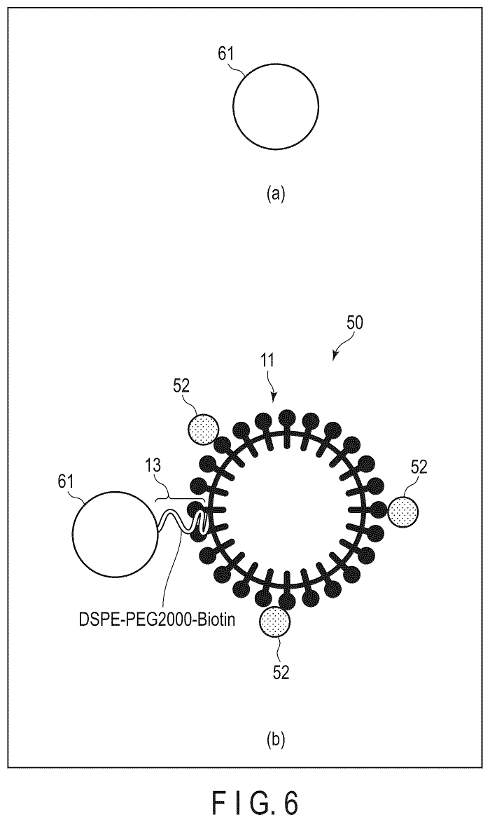

Biodegradable lipid nanoparticles were prepared and streptavidin beads were immobilized against the outside of the lipids of such lipid nanoparticles. Specifically, as shown in part (b) of FIG. 6, cell surface-staying microparticle-bound lipid nanoparticles 50 include biodegradable lipid nanoparticles 11, a linker 13 composed by modification to a portion of the lipid of the lipid nanoparticles 11 (in this lipid nanoparticle, DSPE-PEG2000-Biotin is applicable) and a bead 61 (φ=3 μm, Bang) with streptavidin attached to the surface thereof as a microparticle bound to the linker 13 (part (b) of FIG. 6). The lipid nanoparticles were tested for target cell tropism. The fluorescent material used was Rhodamine-PE (Avanti). Resin beads 61 without lipid nanoparticles bound thereto were prepared as microparticles (part (a) of FIG. 6).

The materials for the biodegradable lipid nanoparticles were FFT-20, DOPE, DOTAP, cholesterol, DSPE-PEG2000-Biotin, and Rhodamine-PE in molar ratios of 31.7:4.5:9.0:51.4:3.4:0.1 respectively. These materials were dissolved in ethanol to obtain a lipid solution. The lipid solution was mixed with the above 10 mM HEPES (pH 7.3) solution using a microflowchip and syringe pump. The mixed solution was further diluted 10-fold with 10 mM HEPES (pH 7.3) and concentrated with an ultrafiltration filter (Amicon Ultra 0.5 Ultracel-50, Merck) to obtain biodegradable lipid nanoparticles 11 with the linker 13. Streptavidin beads were then added in buffer solution to obtain the particle-bound lipid nanoparticles 50 of Example 1.

### Experiment 2: Binding of Biotin and Streptavidin Beads

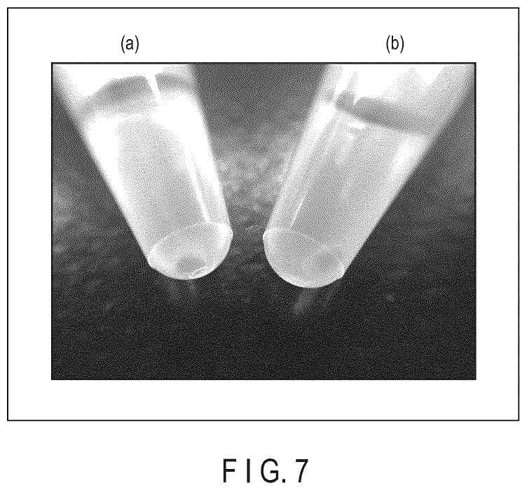

Streptavidin bead suspension (Comparative Example 1) was prepared in microtubes, to which buffer was added as a control and centrifuged. The image obtained by photographing the precipitates is shown in part (a) of FIG. 7. Particle-bound lipid nanoparticles 50 prepared in Experiment 1 (Example 1) were added to the microtubes and centrifuged. The image obtained by photographing the state of the precipitate obtained is shown in part (b) of FIG. 7. Part (b) of FIG. 7 shows that the streptavidin beads had the color of Rhodamine, suggesting that the lipid nanoparticles and streptavidin beads were bound.

### Experiment 3: Observation Under a Microscope

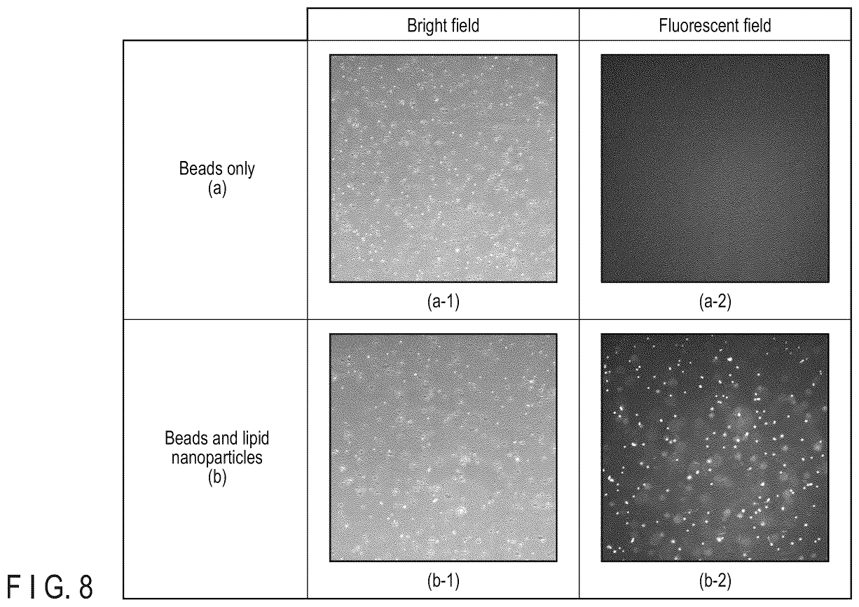

The streptavidin beads of Comparative Example 1 and particle-bound lipid nanoparticles 50 of Example 1 were added to the culture dishes and observed under a microscope in bright field and fluorescent field, respectively. In the bright field, both Comparative Example 1 and Example 1 were observed to be dispersed (FIGS. 8, (a-1) and (b-1)). However, in the fluorescent field of view, only the fluorescent material provided by the particle-bound lipid nanoparticles 50 of Example 1 was observed (part (a-2) and (b-2) of FIG. 8).

### Experiment 4: Target Cell Tropism to Cancer Cells

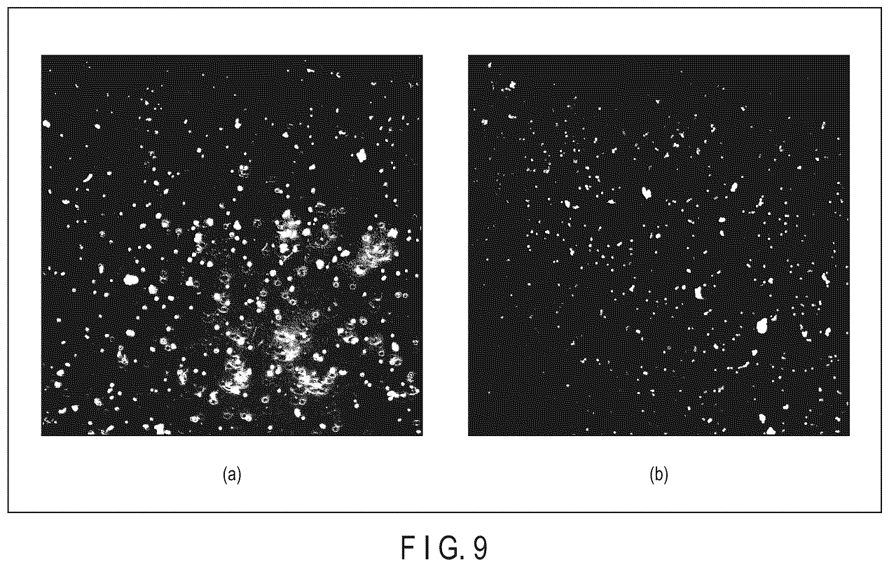

The particle-bound lipid nanoparticles 50 of Example 1 were suspended in buffer solution (composition: pH 7.4 HEPES solution with 200 mM glucose dissolved) and added to two types of giant unilamellar vesicles (GUVs) modeling normal cells and cancer cells, respectively, and the GUVs were observed under a fluorescence microscope after incubation at 37° C. for 10 minutes. Note that, the GUVs of normal cells is formed with a lipid composition containing only DOPC, while the GUVs modeling cancer cells is formed with a lipid composition containing DOPC:DOPS:DOPE in the ratio of 8:1:1. This mimics the lipid compositions of the normal cells and the cancer cells. The results of mixing GUVs and lipid nanoparticles are shown in FIG. 9. It was observed that in normal cell GUVs, the particle-bound lipid nanoparticles 50 and normal cell GUVs were independent of each other (part (a) of FIG. 9). In contrast, the particle-bound lipid nanoparticles 50 were observed to be bound to the surface of the cancer cell GUV (part (b) of FIG. 9).

### Example 2

Next, an example of an experiment in which biodegradable lipid nanoparticles containing a terminally extended linker and, in turn, microparticle-bound lipid nanoparticles consisting of the same biodegradable lipid nanoparticles bound to 1.9 nm microparticles were prepared and the effect on cell transduction was examined will be explained.

### Experiment 5: Preparation of Microparticle-Bound Lipid Nanoparticles

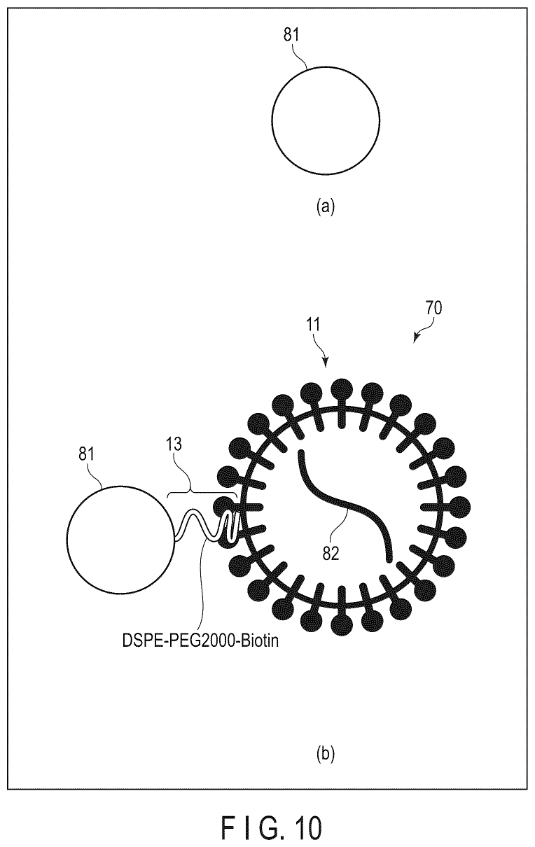

The microparticle-bound lipid nanoparticles of Example 2 differ from the microparticle-bound lipid nanoparticles of Example 1 in the type of cell surface-staying microparticles. Specifically, as shown in part (b) of FIG. 10, microparticle-bound lipid nanoparticle 70 include a biodegradable lipid nanoparticle 11, a nucleic acid 82 encapsulated in the lipid nanoparticle 11, a linker 13 composed by modification to a portion of the lipid comprising the lipid nanoparticle 11, a gold nanoparticle 81 as a nanoparticle attached to the linker 13 (Nanoprobes, Inc., 1.9 nm in diameter) (part (b) of FIG. 10). A comparative example of such microparticle-bound lipid nanoparticles of part (b) of FIG. 10 is biodegradable lipid nanoparticles 11, which do not contain gold nanoparticles and linker (part (a) of FIG. 10).

The method for preparing the microparticle-bound lipid nanoparticles of Example 2 will be described below. First, lipid nanoparticles of the following four compositions (designated as (A), (B), (C), and (D), respectively) were prepared as biodegradable lipid nanoparticles 11. The material for biodegradable lipid nanoparticles 11 of composition (A) was a lipid solution obtained by mixing FFT-10, DOTAP, cholesterol, DMG-PEG2000, DMG-PEG2000-Thiol in ethanol at a mole fraction of 31.7:26.6:9.0:38.0:2.2:0.3, respectively. The material for biodegradable lipid nanoparticles 11 of composition (c) was a lipid solution obtained by mixing FFT-20, DOPE, DOTAP, cholesterol, DMG-PEG2000, and DMG-PEG2000-Thiol in ethanol at a mole fraction of 31.7:4.5:9.0:51.4:3.1:0.3, respectively. In other words, the lipid nanoparticles of composition (A) and composition (C) contain DMG-PEG2000 and DMG-PEG2000-Thiol in a molar ratio of 9:1.

The material for biodegradable lipid nanoparticles 11 of composition (B) was a lipid solution obtained by mixing FFT-10, DOTAP, cholesterol, and DMG-PEG2000 in ethanol at a mole ratio of 31.7:26.6:9.0:38.0:2.5, respectively. The material for biodegradable lipid nanoparticles 11 of composition (D) was a lipid solution obtained by mixing FFT-20, DOPE, DOTAP, cholesterol, and DMG-PEG2000 in ethanol at a mole ratio of 31.7:4.5:9.0:51.4:3.4, respectively. As described above, the compositions (A) and (B) have an equivalent lipid composition base, but composition (B) does not contain DMG-PEG2000-Thiol, i.e., a linker. The lipid nanoparticles of compositions (C) and (D) have an equivalent lipid composition base, but composition (D) does not contain a linker.

Next, a nucleic acid solution was obtained by mixing 1 mg/ml of GFP-mRNA (TriLink BioTechnologies, approx. 1000 bp) and 10 mM HEPES (pH 7.3) solution at a volume ratio of 1:9. The nucleic acid solution was mixed with each of the above four lipid solutions at a ratio of 1:1 using a microflowchip and syringe pump to obtain four types of mixed solutions. In other words, the nucleic acid solution was the same for the above four types of biodegradable lipid nanoparticles 11.

Of the four mixed solutions, for the mixed solution containing biodegradable lipid nanoparticles of composition (A) and composition (C), these mixed solutions were diluted 4-fold with 10 mM HEPES (pH 7.3), then gold nanoparticles (NanoPartz, Inc., diameter 1.8 nm) were added in buffer solution, and allowed to react at room temperature for 30 minutes. On the other hand, the mixed solution containing biodegradable lipid nanoparticles of composition (B) and composition (D) were diluted 10-fold with 10 mM HEPES (pH 7.3) but were not reacted with the gold nanoparticles. Each of the mixed solutions was then concentrated on an ultrafiltration filter (Amicon Ultra 0.5 Ultracel-50, Merck), and from the mixed solution containing biodegradable lipid nanoparticles of composition (A) and composition (C), microparticle-bound lipid nanoparticles 70 were obtained, and from the mixed solution containing biodegradable lipid nanoparticles of composition (B) and composition (D), biodegradable lipid nanoparticles 11 containing nucleic acids 82 were obtained.

### Experiment 6: Introduction of Lipid Nanoparticles into Cancer Cell Lines

A total of four lipid nanoparticles, microparticle-bound lipid nanoparticles from the mixed solution of composition (A) and composition (C), and biodegradable lipid nanoparticles from the mixed solution of composition (B) and composition (D), were introduced into breast cancer cell line MDA-MB231 or human liver cancer-derived cell line Huh7. Specifically, MDA-MB231 cells were seeded in 96 well plates at a cell number of 2×10⁵ and transfected with a drop of each lipid nanoparticle solution equivalent to a total of 200 ng of endogenous nucleic acid. Huh7 cells were seeded in 96 well plates at a cell number of 1×10⁵ and transfected with a drop of each lipid nanoparticle solution equivalent to a total of 100 ng of nucleic acid.

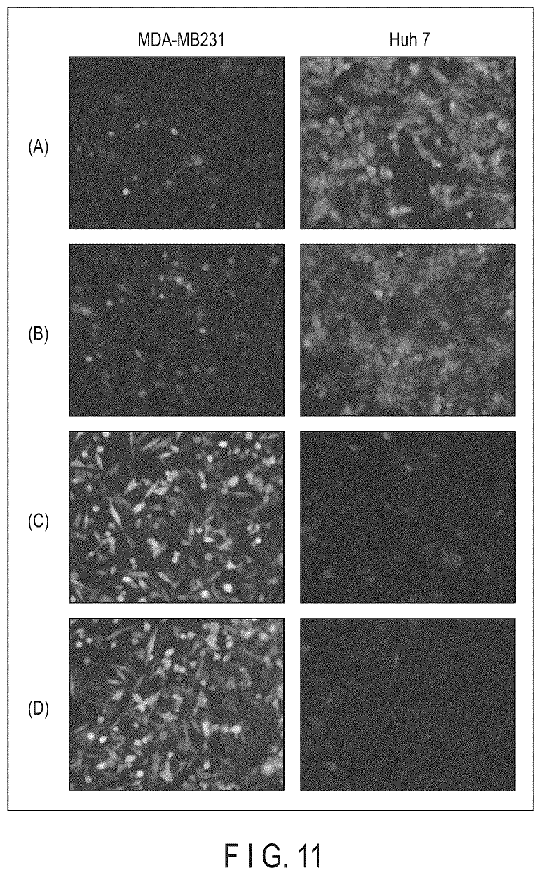

Fluorescence images at 24 hours after transduction are shown in FIG. 11. Similar levels of GFP fluorescence were observed from breast cancer cell lines in both the comparative examples (i.e., compositions (B) and (D)) and the examples (i.e., compositions (A) and (C)), and no differences in shape were observed.

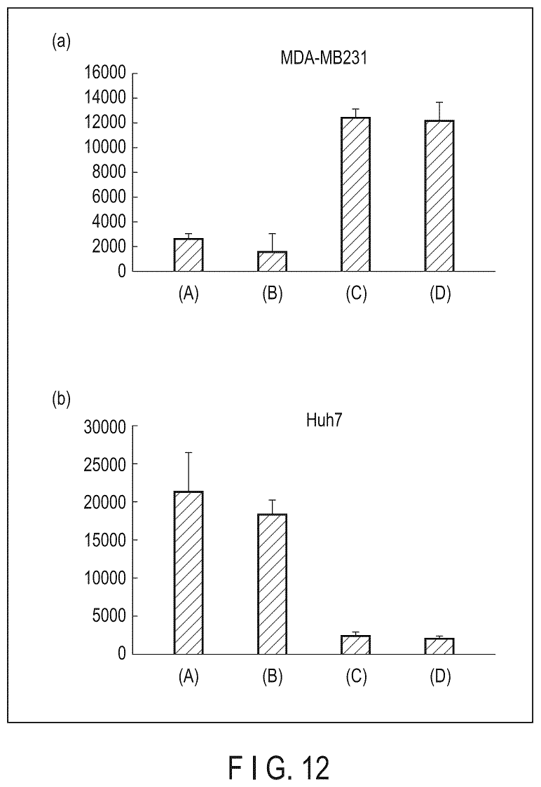

The fluorescence intensity values of GFP measured by a plate reader are shown in FIG. 12. The respective fluorescence intensity values for each lipid nanoparticle composition (A), (B), (C) and (D) were calculated by subtracting the fluorescence intensity value of cells without any lipid nanoparticles (i.e., background) from the fluorescence intensity of cells with each lipid nanoparticle (n=3, error bars indicate standard error). The results of FIG. 12 show that lipid nanoparticles of compositions (A) and (B) were similarly directed toward Huh7, and lipid nanoparticles of compositions (C) and (D) were similarly directed toward MDA-MB231. In other words, the target cell tropism of the lipid nanoparticles is independent of the presence or absence of cell surface staying microparticles such as gold nanoparticles, or in other words, the target cell tropism of the lipid nanoparticles is maintained even when cell surface staying microparticles are bound. In addition, it is suggested that the microparticles can be delivered to desired cells regardless of the type of ionized lipid, such as FFT-10 or FFT-20.

The aforementioned results suggest that lipid nanoparticles with target cell tropism can be utilized to provide cell surface staying microparticle-bound lipid nanoparticles as novel particle-bound lipid nanoparticles for delivering active agents to target cells.

Further examples of embodiment are cited below.

(1) Particle-bound lipid nanoparticle containing a biodegradable lipid nanoparticle, a particle bound to the outer surface of the lipid nanoparticle, and an active substance bound to the surface of the particle.

(2) The particle-bound lipid nanoparticle of (1), wherein the biodegradable lipid nanoparticle contains a linker extending to the end of a portion of its component lipids, and the particle is immobilized on the linker.

(3) The particle-bound lipid nanoparticle of (1) or (2), wherein the active substance acts on the cell surface or on structures present on the cell surface to express an active action.

(4) The particle-bound lipid nanoparticle of any one of (1) to (3), wherein the biodegradable lipid nanoparticle has a lipid composition which exhibits target cell tropism.

(5) The particle-bound lipid nanoparticle of any one of (1) to (4), wherein the particles have the property of remaining on a cell membrane for a certain period of time.

(6) The lipid nanoparticle according to any one of (2) to (5), wherein the linker is contained in a mole fraction of 0.01 to 1% of the component molecules of the biodegradable lipid nanoparticle including the component lipids.

(7) The particle-bound lipid nanoparticle of any one of (1) to (6), wherein the particle is selected from the group consisting of gold, silver, iron oxides, titanium oxides, zinc oxides, silica, lipid nanoparticles, polymer particles, or combinations thereof.

(8) The particle-bound lipid nanoparticle of any one of (1) to (7), wherein the diameter of the particle is 1 nm to 10 μm.

(9) The particle-bound lipid nanoparticle of any one of (1) to (8), wherein the biodegradable lipid nanoparticle encapsulates further active substances or further components within the lipid nanoparticle.

(10) A method for manufacturing a particle-bound lipid nanoparticle of (1), including preparing the biodegradable lipid nanoparticle, mixing the biodegradable lipid nanoparticle with the particle, and mixing the active substance with the resulting mixture to obtain a particle-bound lipid nanoparticle.

(11) The method of (10), wherein the biodegradable lipid nanoparticle contains a linker extending to the end of a portion of the component lipid, and the particle is immobilized to the linker.

(12) A particle-bound lipid nanoparticle manufacturing kit containing a biodegradable lipid nanoparticle, a nanoparticle bound or capable of binding to the outer surface of the lipid nanoparticle, and an active substance.

(13) The particle-bound lipid nanoparticle manufacturing kit of (12), wherein the biodegradable lipid nanoparticle contains a linker extending to the end of a portion of its component lipid, and the particle is immobilizable to the linker.

(14) A combination composition containing a first composition containing a biodegradable lipid nanoparticle and a particle bound to the lipid of the lipid nanoparticle, and a second composition containing an active substance.

(15) The combination composition of (14), wherein the biodegradable lipid nanoparticle contains a linker extending to the end of a portion of its component lipid, and the particle is immobilized to the linker.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A particle-bound lipid nanoparticle comprising:
a biodegradable lipid nanoparticle;
a particle bound to an outer surface of the lipid nanoparticle; and
an active substance bound to a surface of the particle.

2. The particle-bound lipid nanoparticle of claim 1, wherein the biodegradable lipid nanoparticle contains a linker extending to an end of a portion of component lipid thereof and the particle is immobilized to the linker.

3. The particle-bound lipid nanoparticle of claim 1, wherein the active substance interacts with a cell surface or with a structure present on the cell surface and represents an active action.

4. The particle-bound lipid nanoparticle of claim 1, wherein the biodegradable lipid nanoparticle has a lipid composition which exhibits target cell tropism.

5. The particle-bound lipid nanoparticle of claim 1, wherein the particle has property of remaining on a cell membrane for a certain time.

6. The particle-bound lipid nanoparticle of claim 2, wherein the linker is contained in a mole fraction of 0.01 to 1% of component molecules of the biodegradable lipid nanoparticle, including the component lipids.

7. The particle-bound lipid nanoparticle of claim 1, wherein the particle is selected from a group consisting of gold, silver, iron oxides, titanium oxides, zinc oxides, silica, lipid nanoparticles, polymer particles, or a combination thereof.

8. The particle-bound lipid nanoparticle of claim 1, wherein a diameter of the particle is 1 nm to 10 μm.

9. The particle-bound lipid nanoparticle of claim 1, wherein the biodegradable lipid nanoparticle further encapsulates an active substance or a component within a lipid nanoparticle.

10. A method of manufacturing the particle-bound lipid nanoparticle of claim 1, comprising:
preparing the biodegradable lipid nanoparticle;
mixing the biodegradable lipid nanoparticle with the particle; and
mixing an active substance with a resulting mixture to obtain a particle-bound lipid nanoparticle.

11. A particle-bound lipid nanoparticle manufacturing kit, comprising a biodegradable lipid nanoparticle, a particle bound or capable of binding to an outer surface of the lipid nanoparticle, and an active substance.

12. The particle-bound lipid nanoparticle manufacturing kit of claim 11, wherein the biodegradable lipid nanoparticle contains a linker extending to an end of a portion of component lipid thereof, and the particle is immobilizable to the linker.

13. A combination composition comprising: a biodegradable lipid nanoparticle; a first composition containing a particle bound to the lipid of the lipid nanoparticle; and a second composition containing an active substance.
